(19) United States
(12) Patent Application Publication
(10) Pub. No.: US 2024/0315852 A1
(43) Pub. Date: Sep. 26, 2024

(54) BONE GROWTH ENHANCEMENT CONTAINMENT DEVICE

(71) Applicant: CTL Medical Corporation, Addison, TX (US)

(72) Inventors: Lance TIGYER, Dayton, OH (US); Jon SUH, Ambler, PA (US); Sean SUH, Milltown, NJ (US)

(21) Appl. No.: 18/611,512

(22) Filed: Mar. 20, 2024

Related U.S. Application Data
(60) Provisional application No. 63/453,365, filed on Mar. 20, 2023.

(51) Int. Cl. A61F 2/44; A61F 2/28; A61F 2/30

(52) U.S. Cl. CPC A61F 2/4455; A61F 2002/2839; A61F 2002/285; A61F 2002/30593; A61F 2310/00353

(57) ABSTRACT

Disclosed are devices, systems and method for inserts and/or other containment devices which can be incorporated into implants that facilitate, promote and/or accelerate the growth of bone in and/or through a region of interest, such as in a spinal segment of a patient undergoing spinal fusion surgery.

### CROSS-REFERENCE TO RELATED APPLICATIONS

This application claims the benefit of U.S. Provisional Application No. 63/453,365 entitled “BONE GROWTH ENHANCEMENT CONTAINMENT DEVICE” filed Mar. 20, 2023, the disclosure of which is incorporated by reference herein in its entirety.

### FIELD OF THE INVENTION

The present subject matter relates generally to implants and related devices used in surgeries of the human body, including orthopedic implants such as joint and/or bone replacement implants used in in spinal surgeries, dental surgeries and/or other orthopedic procedures. More specifically, such implants can incorporate containment devices for facilitating, promoting and/or accelerating the growth of bone in and/or through a region of interest, such as in a spinal segment of a patient undergoing spinal fusion surgery. In some embodiments, disclosed are low-cost design(s) for containment devices and/or bone growth enhancement materials, particularly Si3N4 or other materials, that be added and/or retro-fitted to any bone fusion cage with a bone graft window or other orthopedic devices.

### BACKGROUND OF THE INVENTION

The spinal column of vertebrates provides support to bear weight and protection to the delicate spinal cord and spinal nerves. The spinal column includes a series of vertebrae stacked on top of each other. There are typically seven cervical (neck), twelve thoracic (chest), and five lumbar (low back) segments. Each vertebra has a cylindrical shaped vertebral body in the anterior portion of the spine with an arch of bone to the posterior, which covers the neural structures. Between each vertebral body is an intervertebral disk, a cartilaginous cushion to help absorb impact and dampen compressive forces on the spine. To the posterior, the laminar arch covers the neural structures of the spinal cord and nerves for protection. At the junction of the arch and anterior vertebral body are articulations to allow movement of the spine.

Various types of problems can affect the structure and function of the spinal column. These can be based on degenerative conditions of the intervertebral disk or the articulating joints, traumatic disruption of the disk, bone or ligaments supporting the spine, tumor or infection. In addition, congenital or acquired deformities can cause abnormal angulation or slippage of the spine. Anterior slippage (spondylolisthesis) of one vertebral body on another can cause compression of the spinal cord or nerves. Patients who suffer from one or more of these conditions often experience extreme and debilitating pain and can sustain permanent neurological damage if the conditions are not treated appropriately.

Various physical conditions can manifest themselves in the form of damage or degeneration of an intervertebral disc, the result of which is mild to severe chronic back pain. Intervertebral discs serve as “shock” absorbers for the spinal column, absorbing pressure delivered to the spinal column. Additionally, they maintain the proper anatomical separation between two adjacent vertebrae. This separation is necessary for allowing both the afferent and efferent nerves to exit and enter, respectively, the spinal column. Alternatively, or in addition, there are several types of spinal curvature disorders. Examples of such spinal curvature disorders include, but need not be limited to, lordosis, kyphosis and scoliosis.

One technique of treating spinal disorders, in particular the degenerative, traumatic and/or congenital issues, is via surgical arthrodesis of the spine. This can be accomplished by removing the intervertebral disk and replacing it with implant(s) and/or bone and/or immobilizing the spine to allow the eventual fusion or growth of the bone across the disk space to connect the adjoining vertebral bodies together. The stabilization of the vertebra to allow fusion is often assisted by the surgically implanted device(s) to hold the vertebral bodies in proper alignment and allow the bone to heal, much like placing a cast on a fractured bone. Such techniques have been effectively used to treat the above-described conditions and in most cases are effective at reducing the patient's pain and preventing neurological loss of function.

Complications of joint fusions and/or other procedures of the spine can include those applicable to any surgery such as bone and/or soft-tissue infection, wound dehiscence, and failure of fixation. Other complications which may be more specific to fusion procedures can include malalignment, proximal or distal joint deterioration, and delayed union or nonunion, including potential complications resulting from medical comorbidities, patient noncompliance, and/or inappropriate fixation. Accordingly, there is need for further improvement in surgical implants, and the present subject matter is such improvement.

### BRIEF SUMMARY OF THE INVENTION

The following presents a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

Various embodiments herein disclose an insert or containment device for facilitating, promoting and/or accelerating the growth of bone in and/or through a region of interest, such as in a spinal segment of a patient undergoing spinal fusion surgery. In some embodiments, disclosed are low-cost design(s) for containment devices and/or bone growth enhancement materials, particularly Si3N4 or other materials, that can be particularized and/or retro-fitted to any bone fusion cage having a bone graft window or similar orthopedic devices. In accordance with various aspects of the present subject matter, containment devices or “inserts” are disclosed and described what may facilitate, promote and/or accelerate the growth of bone in and/or through a region of interest, such as in a spinal segment of a patient undergoing spinal fusion surgery. In various embodiments, such inserts devices can comprise and/or incorporate various additives, including but not limited to silicon nitride (i.e., Si3N4 and/or chemical analogues thereof) or other material additives in their construction, either distributed throughout the entirety of the insert as well as within openings, components, portions, layers and/or surfaces thereof. In various embodiments, one or more inserts can be placed within and/or incorporated into an implant, with the silicon nitride or other material additives therein providing a variety of improvements to the implant, such as where the additives may be highly osteo-inductive and/or osteoconductive and/or will desirably facilitate and/or promote implant fixation to adjacent living bone surfaces and/or may reduce and/or inhibit periprosthetic infection and/or bacterial adhesion to the surfaces and/or interior portions of the implant.

In various applications, existing commercially available implant designs and/or the performance thereof may be enhanced by the addition of one or more inserts incorporating as silicon nitride or other material additives, including the use silicon nitride implant components as well as one or various combinations of titanium, chrome cobalt, stainless steel, silicone, poly (ether ether ketone) (PEEK), ultra-high molecular-weight polyethylene (UHMWPE), polyurethane foams, polylactic acid, apatites and/or materials in combination with various additives, including the use with various 3D printable materials. In such cases, the employment of such materials and/or material mixtures in an insert construction and/or composition may enhance the strength and/or durability of a desired implant design, as well as allow for improved surgical outcomes and/or greatly reduced complication rates.

In various embodiments described herein, a variety of manufacturing steps and/or processes may be performed to create a desired insert and/or component(s) thereof, including the use of different manufacturing processes to create a single insert and/or the employment of multiple different manufacturing processes to create inserts that may be ultimately assembled into a single implant. Such processes could include any combinations of one or more of the following: (1) casting, (2) molding (including injection molding), (3) subtractive machining (i.e., milling and drilling), (4) additive machining (i.e., additive 3D printing), (5) surface coating and sputtering, (6) surface embedding, and/or any combinations thereof.

If desired, an insert could be constructed from a variety of modular components, including modular components comprising different materials. If desired, such modular components could be provided in a kit form for selection and/or assembly in a surgical theatre and/or in situ during a surgical procedure. If desired, various components may be removable and replaceable.

In accordance with various aspects of the present subject matter, insert materials capable of being molded and/or processed using other manufacturing methods can be combined with a variety of additives, including (but not limited to) silicon nitride (i.e., Si3N4 and/or chemical analogues thereof) in their mixtures and/or composition, which may include the incorporation of silicon nitride powders, granules, particulates, portions, pebbles, blocks, layers and/or coatings of solids and/or particulates within the insert and/or the insert material mixture. In various embodiments, an exemplary insert material mixture may comprise a liquid, powder, granular substance, paste, gel or dough, and in various embodiments may preferably harden and/or cool to a substantially solid solidified material after processing.

In at least one alternative embodiment, an insert material such as poly (ether ether ketone) or “PEEK” material can be formed and then openings within the insert can be filled with various percentages by weight and/or volume of a ceramic material such as a silicon nitride material, which when combined can result in an insert and/or other structure capable of insertion into an interbody implant for implantation in a bony defect and/or other location. In various embodiments, the ceramic material may comprise a granular or regularly/irregularly shaped material, with the granules having a plurality of interconnecting micropores. In various embodiments, a plurality of different sizes of granules may be used.

In at least one alternative embodiment, an insert can be filled or otherwise infused with a “filler” comprising various percentages by weight and/or volume of a powdered, granulated and/or fluidized silicon nitride material, which will desirably remain within the insert (optionally hardening and/or curing within the insert). The insert can then be introduced into the implant in a desired manner. In various embodiments, the silicon nitride material(s) and/or other materials will be highly osteo-inductive and/or osteoconductive and will desirably facilitate and/or promote fixation to adjacent living bone surfaces, while concurrently reducing and/or inhibiting periprosthetic infection and/or bacterial adhesion.

If desired, implants can be constructed from a variety of modular components, including modular insert components comprising different materials in combination with injectable or formable silicon nitride. If desired, such modular components could be provided in a kit form for selection and/or assembly in a surgical theatre and/or in situ during a surgical procedure. If desired, various components may be removable and replaceable.

Various surgical methods for preparing anatomical surfaces and/or for implanting or placement of the various devices and/or components described herein are also described, including the insertion and placement of implants between adjacent vertebrae of the spine as well as within bones and/or between bones and/or joint surfaces or other body locations.

In accordance with another aspect of the present subject matter, various methods for manufacturing inserts and/or other devices and/or components thereof, as set for within any of the details described with the present application, are provided.

While embodiments and applications of the present subject matter have been shown and described, it would be apparent that other embodiments, applications and aspects are possible and are thus contemplated and are within the scope of this application.

The following description and the annexed drawings set forth in detail certain illustrative aspects of the subject matter. These aspects are indicative, however, of but a few of the various ways in which the principles of the subject matter may be employed and the present subject matter is intended to include all such aspects and their equivalents. Other objects, advantages and novel features of the subject matter will become apparent from the following detailed description of the subject matter when considered in conjunction with the drawings.

### BRIEF DESCRIPTION OF THE SEVERAL VIEWS OF THE DRAWINGS

The foregoing and other features and advantages of the present subject matter will become apparent to those skilled in the art to which the present subject matter relates upon reading the following description with reference to the accompanying drawings. It is to be appreciated that two copies of the drawings are provided; one copy with notations therein for reference to the text and a second, clean copy that possibly provides better clarity.

FIG. 1 depicts one exemplary embodiment of a height adjustable mating containment capsule or insert for use with a variety of implant designs;

FIG. 2 depicts the assembled insert with various height instances of FIG. 1, filled with a flowable and/or malleable material;

FIGS. 3A and 3B depict filled inserts being introduced into an interbody implants;

FIG. 4A depicts a top plan view of the plurality of filled inserts in the interbody implant of FIG. 3;

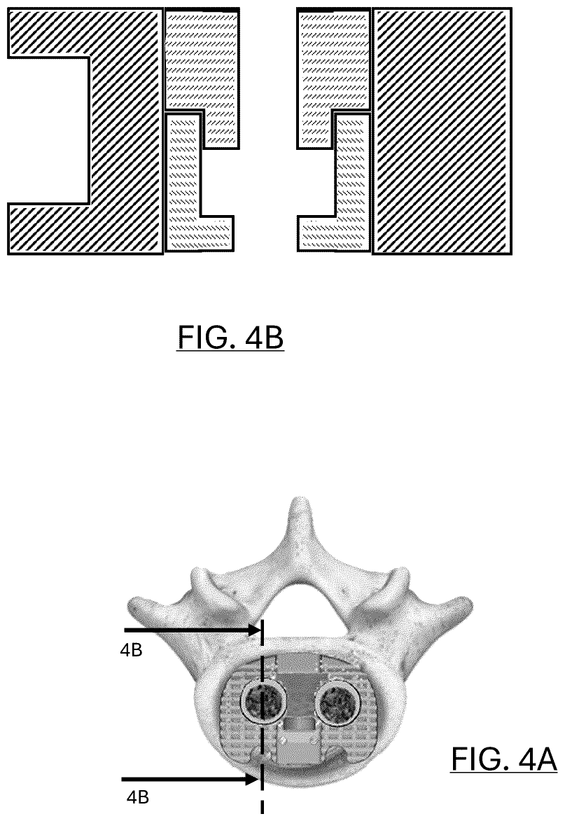

FIG. 4B depicts a cross-sectional view of the interbody implant and height adjustable insert of FIG. 4A taken along line 4B-4B;

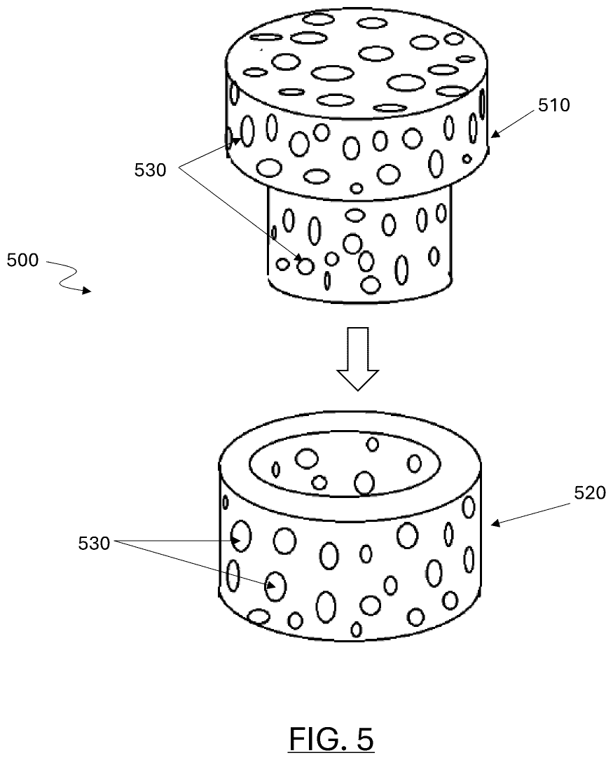

FIG. 5 depicts one alternative embodiment of a containment capsule or insert for use with a variety of implant designs; and

FIGS. 6A and 6B depict another alternative embodiment of a containment capsule or insert for use with a variety of implant designs or other insert designs.

### DETAILED DESCRIPTION OF THE INVENTION

The disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure. Moreover, it is noted that like reference numerals may represent similar parts throughout the several views of the drawings. In addition, the following is a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

In various embodiments, the terms “including,” “comprising” and variations thereof, as used in this disclosure, should be interpreted as “including, but not limited to,” unless expressly specified otherwise. The terms “a,” “an,” and “the,” as used in this disclosure, mean “one or more,” unless expressly specified otherwise.

In some embodiments, devices and/or device components that may be disclosed in communication with each other need not necessarily be in continuous communication with each other, unless expressly specified otherwise. In addition, components that are in direct contact with each other may contact each other directly or indirectly through one or more intermediary articles or devices. The device(s) disclosed herein may comprise a material such as a silicon nitride block, putty or powder, which may alternatively be combined, in various embodiments, with other insert materials such as, for example, a polymer, a metal, an alloy, or the like. For instance, a disclosed device(s) may comprise a silicon nitride material, alone or in combination with a Polyether Ether Ketone (PEEK), a titanium, a titanium alloy, or the like, or various combinations of the foregoing. The material may be formed by a process such as, for example, an active reductive process of a metal (e.g., titanium or titanium alloy) to increase the amount of nanoscaled texture to device surface(s), so as to increase promotion of bone growth and fusion.

Although process steps, method steps, or the like, may be described in a sequential order, such processes and methods may be configured in alternate orders. In other words, any sequence or order of steps that may be described does not necessarily indicate a requirement that the steps be performed in that order. The steps of the processes or methods described herein may be performed in any order practical. Further, some steps may be performed simultaneously.

When a single component, device and/or article is described herein, it will be readily apparent that more than one component, device and/or article may be used in place of a single component, device and/or article. Similarly, where more than one component, device and/or article is described herein, it will be readily apparent that a single component, device and/or article may be used in place of the more than one component, device and/or article. The functionality or the features of a component, device and/or article may be alternatively embodied by one or more other components, devices and/or articles which are not explicitly described as having such functionality or features.

Certain exemplary embodiments will now be described to provide an overall understanding of the principles of the structure, function, manufacture, and use of the components, devices and methods disclosed herein. One or more examples of these embodiments are illustrated in the accompanying drawings. Those of ordinary skill in the art will understand that the components, devices and/or methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention provides various components, devices, systems and methods for treating various anatomical structures of the spine and/or other areas of human and/or animal bodies. While the disclosed embodiments may be particularly well suited for use during surgical procedures for the repair, fixation and/or support of vertebrae, it should be understood that various other anatomical locations of the body may benefit from various features of the present invention, including for the repair of bones and for use in, for example, orthopedic surgery, including vertebrae repair, musculoskeletal reconstruction, fracture repair, hip and knee reconstruction, osseous augmentation procedures and oral/maxillofacial surgery.

Various embodiments herein encompass inserts that incorporate a block, flowable and/or moldable silicon nitride (i.e., Si3N4 and/or chemical analogues thereof) and/or other material additives in their mixtures and/or composition, which may include the incorporation of silicon nitride slurries, powders, granules, particulates, portions, pebbles, blocks, layers and/or coatings of solids and/or particulates within the material mixture.

In at least one exemplary embodiment, an insert could comprise a standard or commonly-accepted implant material, optionally silicon nitride or other medical materials, which is then filled or otherwise infused with varying amounts of a flowable and/or moldable material, such as a silicon nitride material, allograft and/or autograft (among others), which when combined can subsequently be inserted into an implant such as a spinal interbody implant.

In various embodiments, inserts can be formed and/or manufactured which incorporate silicon nitride and/or other materials therein, which materials may facilitate bone ingrowth, bone outgrowth and/or bone through-growth in varying amounts. An implant then “fitted” with such inserts would desirably benefit from the improved bacteriostatic properties of silicon nitride and/or other materials, and will further allow for superior adhesion and/or anchoring of the implant to surrounding structures. In various embodiments, the properties of the disclosed inserts will desirably provide improvements to the overall implant performance, including flexibility in manufacturing and structural diversity, an ability to incorporate “weaker” materials such as fluidized silicon nitride and/or other materials into strong, tough and/or reliable implant constructs, improved imaging characteristics, the inclusion of hydrophilic surfaces and/or structures into implants, the inclusion of osteoconductive and/or osteoinductive materials in the insert, and/or the provision of anti-bacterial characteristics.

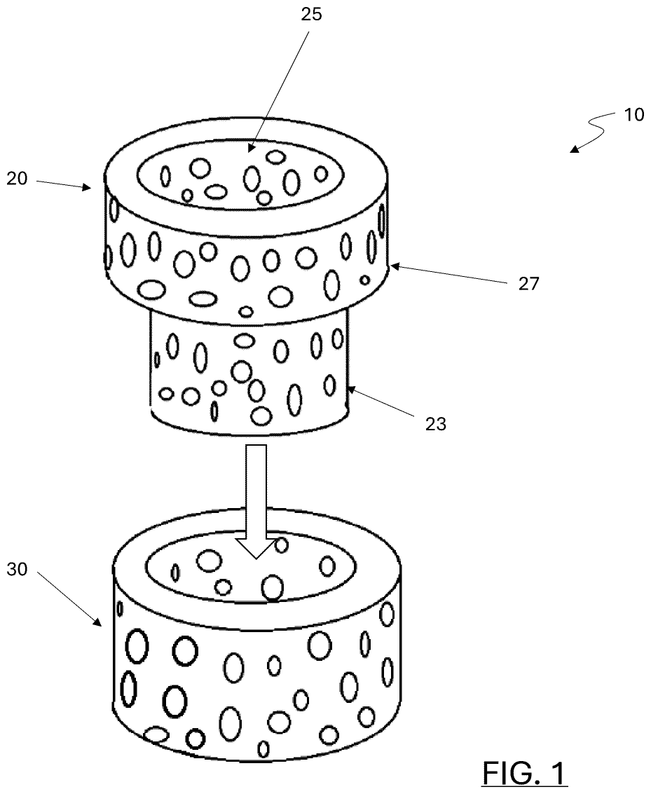

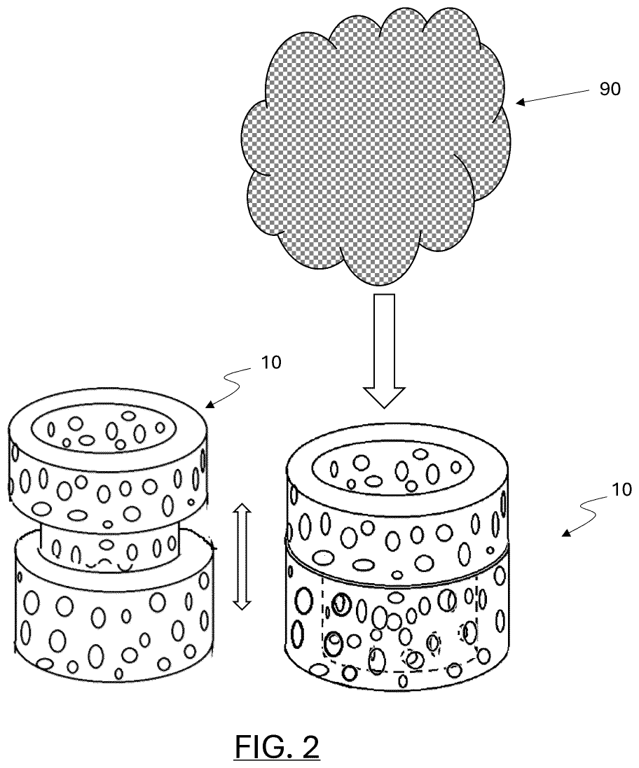

FIG. 1 depicts one exemplary embodiment of a containment capsule or insert 10 for use with a variety of implant designs. In this embodiment, the insert 10 comprises an upper body 20 and a lower body 30, the upper body 20 comprising a generally hollow upper cylindrical tube 23 having an upper bore 25 therein and an upper flanged section 27, the lower body 30 comprising a lower cylindrical tube section 33 having a lower bore 25 therein. Desirably, the lower bore 25 can be sized and configured to allow a lower portion of the generally hollow upper cylindrical tube 23 to slide into the lower bore 25, to create an assembled insert assembly of various height ranges (see FIG. 2).

In various embodiments, a containment device or insert may comprise a two- or more part cylindrical body or other slidably fitting geometries having generally flattened ends, wherein the body could be sized such that the flat ends of the cylindrical body are coplanar with the upper and lower plates of the implant in which it is contained. The device can optionally be sized substantially smaller in cross-section than the opening(s) in which it is contained, with additional material (e.g., bone growth inducing material such as autograft/allograft and/or bone growth enhancement materials such as Si3N4) placed between the outer walls of the cylindrical body and the inner walls of the opening in which it is placed. Of course, component shapes other than cylindrical shapes are contemplated for alternative designs herein.

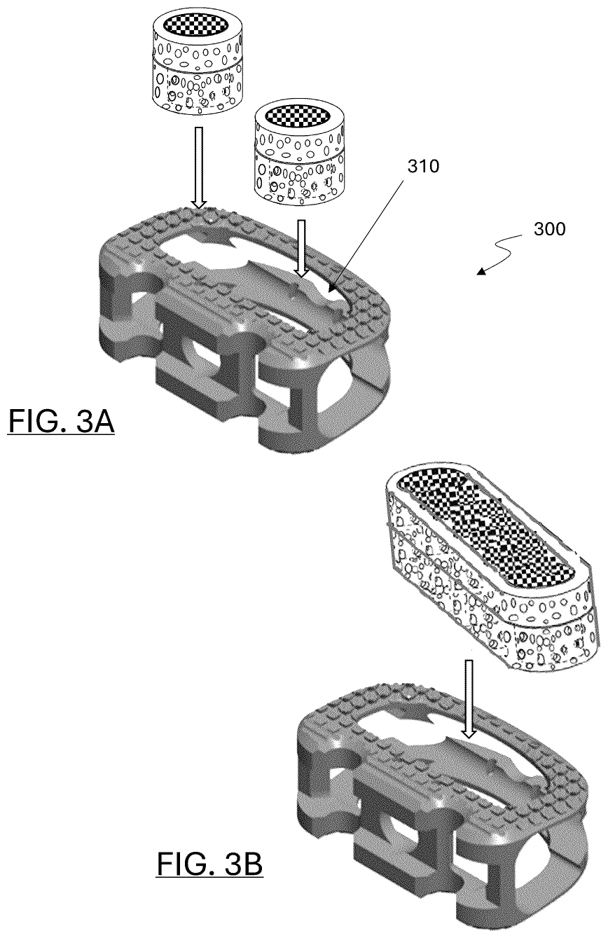

Once assembled, a containment device may be packed with a filler material and inserted into an intervertebral implant, and subsequently inserted into a disc space in a known manner. As shown in FIG. 2, a flowable or moldable material 90, including but not limited to a ceramic material such as silicon nitride, may be filled or otherwise infused into the insert 10, which activity may be accomplished prior to, during and/or after assembly of the individual insert components. As depicted in FIG. 3, one or more of the filled inserts 300 can then be placed into a graft chamber 310 (or plurality of graft chambers) of an intervertebral cage 300 or similar implant. If desired, the insert may be sized and configured to occupy substantially the entirety of the graft chamber, or the inset may be smaller than the graft chamber. In various embodiments, the insert or containment device will be significantly smaller and/or differently shaped than the inner walls of the graft opening in the implant.

If the insert is substantially smaller than one or more dimensions of the graft chamber in which it sits, any additional space between the opening within the implant and the insert(s) can be filled with a first and/or second filler, which second filler may comprise the same materials as the first filler, or may be one or more different materials. If desired, the first filler can comprise a liquid, slurry or putty containing Si3N4 and other materials (as well as mixtures thereof). In some embodiments. The insert material may comprise a standard implant material such as titanium, PEEK or similar materials, while in other embodiments the insert material may comprise a solid and/or modular block of Si3N4 or similar materials. In various embodiments, the first filler material may be the same or different material as the second filler material, where the first filler (or second filler) may optionally be of a different viscosity or state of matter as the second filler (or first filler).

In one exemplary embodiment, a supplementary containment device or insert may incorporate design features that allows one or more (e.g., a plurality or two or three or more) containment devices to be utilized with virtually any fusion cages in the market which have a central window (or multiple windows or openings therein) or similar openings that can be filled with a BMP and/or bone graft material and/or anti-inflammation medications.

For example, a lumbar cage may come in the following dimensions:
Width: 7˜ 40 mm in at least 1 mm increments.
Length: 20˜ 65 mm in at least 2 mm increments
Height: 5˜ 24 mm in at least 1 mm increments
Lordosis: 0˜ 30 deg in at least 1 degree lordosis

An exemplary containment device can desirably comprise a metal, polymer (e.g., PEEK), ceramic and/or other acceptable and/or osteo-integrative materials, which can be utilized to construct the various geometry shaped cages. The containment device can optionally comprise a multi-piece assembly, with various sized and/or shaped pieces being provided in a kit form, wherein the various pieces are compatible to allow for assembly and creation of various shaped and/or sized containment devices. In various embodiment, the disclosed insert devices could be useful with all CTL cage family and competitive cage designs having bone graft windows or similar structures. Suitable devices could comprise virtually any implantable materials including, but not limited to, PEEK, PEKK, TITANIUM and its ALLOYS, SS316-L, CHROMIUM, etc.

In some embodiments, a plurality of devices may be inserted into a single cage or other implant, wherein the devices may be in contact with each other and/or various inner walls of the opening and/or spaced apart from each other and/or the inner walls within the implant space.

Silicon nitride (Si3N4) and its various analogs can impart both antibacterial and osteogenic properties to an implant, including to incorporating fillers and/or coated with layers of Si3N4 of varying thicknesses. In bone replacement as well as prosthetic joint fusion and/or replacement, osseous fixation of implants through direct bone ingrowth (i.e., cementless fixation) is often preferred, and such is often attempted using various surface treatments and/or the incorporation of porous surface layers (i.e., porous Ti6Al4V alloy) on one or more bone-facing surfaces of an implant. Silicon nitride surfaces and/or interior portions express reactive nitrogen species (RNS) that promote cell differentiation and osteogenesis, while resisting both gram-positive and gram-negative bacteria. This dual advantage of RNS in terms of promoting osteogenesis, while discouraging bacterial proliferation, can be of significant utility in a variety of implant designs.

Desirably, the inclusion of silicon nitride components into the disclosed inserts can encompass the use of flowable compositions, as well as use of granularized and/or powdered silicon nitride. If desired, one of more of the insert components themselves may be formed from bulk silicon nitride, as well as other materials that may also include silicon nitride components and/or layers therein, with the silicon nitride becoming an active agent of bone fusion. RNS such as N2O, NO, and —OONO are highly effective biocidal agents, and the unique surface chemistries of Si3N4 facilitate its activity as an exogenous NO donor. Spontaneous RNS elution from Si3N4 discourages surface bacterial adhesion and activity, and unlike other direct eluting sources of exogenous NO, Si3N4 elutes mainly NH4+ and a small fraction of NH3 ions at physiological pH, because of surface hydrolysis and homolytic cleavage of the Si—N covalent bond. Ammonium NH4+ can enter the cytoplasmic space of cells in controlled concentrations and through specific transporters, and is a nutrient used by cells to synthesize building-block proteins for enzymes and genetic compounds, thus sustaining cell differentiation and proliferation. Together with the leaching of orthosilicic acid and related compounds, NH4+ promotes osteoblast synthesis of bone tissue and stimulates collagen type 1 synthesis in human osteoblasts. Conversely, highly volatile ammonia NH3 can freely penetrate the external membrane and directly target the stability of DNA/RNA structures in bacterial cells. However, the release of unpaired electrons from the mitochondria in eukaryotic cells activates a cascade of consecutive reactions, which starts with NH3 oxidation into hydroxylamine NH2OH (ammonia monooxygenase) along with an additional reductant contribution leading to further oxidation into NO2-nitrite through a process of hydroxylamine oxidoreductase. This latter process involves nitric oxide NO formation. In Si3N4, the elution kinetics of such nitrogen species is slow but continuous, thus providing long-term efficacy against bacterial colonies including mutants (which, unlike eukaryotic cells, lack mitochondria). However, when slowly delivered, NO radicals have been shown to act in an efficient signaling pathway leading to enhanced differentiation and osteogenic activity of human osteoblasts. Desirably, Si3N4 materials can confer resistance against adhesion of both Gram-positive and Gram-negative bacteria, while stimulating osteoblasts to deposit more bone tissue, and of higher quality.

Desirably, the disclosed inserts comprise implant material mixtures which grant improved structural properties to the inserts in combination with improved osteoconductivity to regenerate and heal the host bone tissue provided by the filer(s). In such embodiments, the insert material will desirably provide improved structural properties, whilst the flowable and/or moldable ceramic filler retains the insert within the implant and/or allows host tissue to bind and regenerate around and within the filler material(s).

Once implanted in a desired location, an implant incorporating insert(s) with silicon nitride fillers will desirably be highly osteo-inductive and/or osteoconductive and will desirably facilitate and/or promote fixation of the implant to adjacent living bone surfaces, while concurrently reducing and/or inhibiting periprosthetic infection and/or bacterial adhesion to the surfaces and/or interior portions of the implant.

In accordance with another aspect of the present subject matter, various methods for manufacturing an implant comprising a material mixture including silicon nitride and/or other materials, as set for within any of the details described with the present application, are provided.

As previously noted, the various implants and/or inserts thereof can incorporate a silicon nitride material (i.e., Si3N4 and/or chemical analogues thereof) in their composition, either in the entirety of the implant as well as components, portions, layers, fillings, dispersed particulates, fillers and/or surfaces thereof. The incorporation of silicon nitride as a component material for spinal or other implants can provide significant improvements over existing implant materials and material designs currently available, as the silicon nitride material(s) will desirably be highly osteo-inductive and/or osteoconductive and will facilitate and/or promote implant fixation to adjacent living bone surfaces, while concurrently reducing and/or inhibiting periprosthetic infection and/or bacterial adhesion to the surfaces and/or interior portions of the implant. In various embodiments, materials including silicon nitride materials of differing compositions and/or states (i.e., solid, liquid and/or flowable or moldable “slurry” states, for example) could be utilized in a single implant and/or portions thereof, including the use of a moldable silicon nitride “paste” placed within an insert in the centrally positioned “graft chamber” of the implant. If desired, an implant could include some portion or insert formed from a silicon nitride material, wherein the silicon nitride or similar component could extend completely through an implant, or only extend partially into and/or out of an implant.

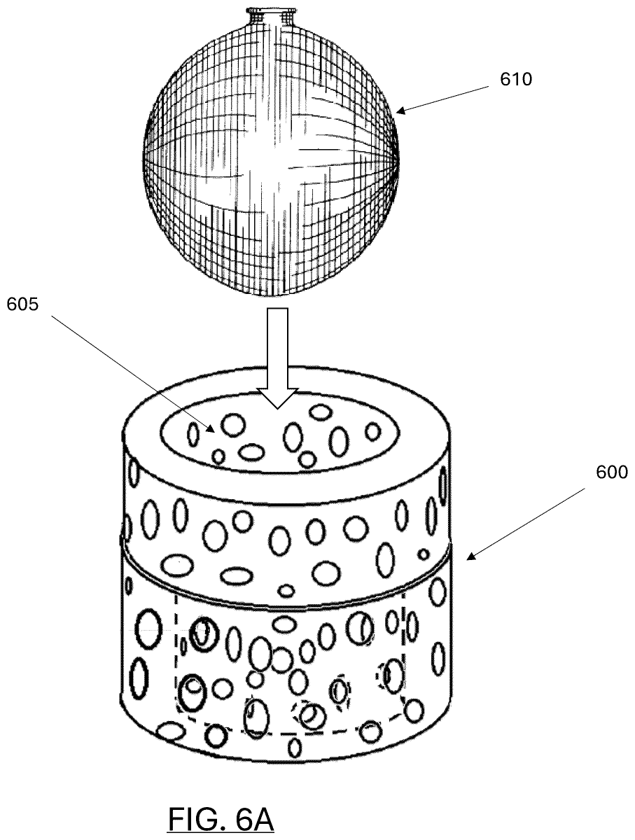

In some embodiments, an insert may be formed from a monolithic block of silicon nitride, while in others the insert could be filled with a putty or gel containing silicon nitride, or various combinations thereof. In one exemplary embodiment, an insert can comprise a PEEK or titanium hollow cylindrical body, with the cylinder filled with bone graft or other materials, including a silicon nitride putty, if desired. In alternative embodiments, such as depicted in FIG. 5, the insert 500 can comprise a solid upper plug 510 and a hollow, lower plug 520 which can be assemble together into the insert. In such an embodiment, a filler material may be introduced into pores 530 within the upper and lower plug material. Alternatively, one or both of the upper and lower plugs may comprise a ceramic material such as silicon nitride.

In various embodiments, a silicon nitride filled insert may display varying degrees of hydrophobicity for various medical grade materials, including silicon nitride in various forms. Silicon nitride can be much less resistant to water penetration than other materials, which can be a highly desirably characteristic in many applications. If desired, an implant incorporating a silicon nitride filled insert can induce neovascularization within the porous sections of the implant, including internal pores colonized with mineralized bone to depths exceeding 5.5 mm or more.

Applicant proposes that silicon nitride filled inserts can be particularly useful in alleviating various limitations inherent with existing implant materials. In many instances, a pure PEEK implant may often be accompanied by surgical bone defects that do not fill in with new bone over time, as well as potential infection sites proximate to the implant that may be difficult or impossible to resolve (potentially necessitating implant removal in some cases). In a similar manner, bone infection sites near titanium implants can also be difficult or impossible to resolve, and may similarly necessitate implant removal. However, with a mixed material implant incorporating a silicon nitride filled insert, the surface chemistry of the implant can actively destroy infectious bacterial agents, and also induce new bone growth immediately upon implantation. In essence, the effect of the silicon nitride filler material on new bone growth may act like a magnet on ferrous materials, actively “drawing” new bone near and into the implant.

Another significant advantage of using silicon nitride filled inserts in bone implants is the anti-bacterial effects of the material on infectious agents. Upon implantation, a silicon nitride filler can induce an inflammatory response action which attacks bacterial biofilms near the implant. This reaction can also induce the elevation of bacterial pods above the implant surface by fibrin cables. Eventually the bacteria in the vicinity of the silicon nitride filler surfaces will be cleared by macrophage action, along with the formation of osteoblastic-like cells. In various experiments involving comparisons between standard implants and implants with silicon nitride filled inserts, cell viability data in show the existence of a larger population of bacteria on the standard medical materials as compared to Si3N4 filled implants. A statistically validated decreasing trend for the bacterial population with time can be detected on implants, with a highest decrease rate on Si3N4 filled substrates. Moreover, the fraction of dead bacteria at 48 hours can be negligible on the standard implants, while almost the totality of bacteria will undergo lysis on the Si3N4 filled substrates. In addition, optical density data can provide a direct assessment of the high efficacy of the Si3N4 surfaces in reducing bacterial adhesion.

In various embodiments, the disclosed designs can provide various combinations of significant advantages and desirable attributes of an abiotic spinal spacer or similar implant, such as one or more of the following: biocompatibility, mechanical integrity, radiological traceability, osteoconductivity, osteoinductivity and/or bacteriostasis. In various embodiments, silicon nitride insert materials and/or filling thereof can be incorporated into a variety of implants and implant-like materials, including (1) orthopedic bone fusion implants (i.e., screws, cages, cables, rods, plugs, pins), (2) dental implants, (3) cranial/maxillofacial implants, (4) extremity implants, (5) hip and joint implants, (6) bone cements, powders, putties, gels, foams, meshes, cables, braided elements, and (7) bone anchoring elements and/or features.

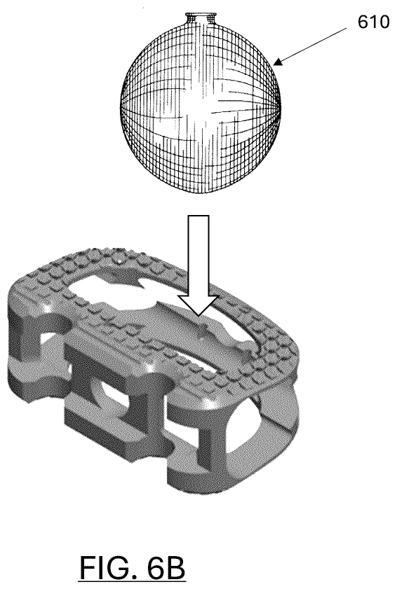

FIG. 6 depicts another alternative embodiment of a filler material for use with a containment capsule or insert 600. In this embodiment filler material can be contained within a flexible mesh or bag 610, which desirably fills and forms to the surrounding opening 605 within the insert 600. A wide variety of materials, including morselized bone and/or silicon nitride can be placed within the bag, and then the bag can be pushed into the opening 605. Alternatively, the bag may be placed within the opening, and then the selected filler material(s) (including silicon nitride, if desired) can be subsequently introduced into the bag within the graft cavity.

While embodiments and applications of the present subject matter have been shown and described, it would be apparent that other embodiments, applications and aspects are possible and are thus contemplated and are within the scope of this application. The subject matter, therefore, is not to be restricted except in the spirit of the appended claims.

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

As previously noted, the use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A containment device for placement in a graft chamber of an interbody cage for implantation between two vertebrae, comprising
a containment body sized and configured to be inserted fully within the graft chamber, the containment body being height adjustable.

2. The containment device of claim 1, wherein the containment body comprises a plurality of adjustable components.

3. The containment device of claim 1, wherein the containment body comprises a first, non-flowable material, the containment body having at least one opening therein, and
a second, flowable material positioned within the at least one opening.

4. The containment device of claim 1, wherein the containment body occupies less than the volume of the graft chamber.

5. The containment device of claim 4, wherein a remaining portion of a volume of the graft chamber not occupied by the containment body is filled with a second bone growth enhancement material which retains the containment device within the graft chamber.

6. The containment device of claim 1, wherein the containment body includes a first generally planar upper surface which engages with a first bone surface of a first vertebral body and a second generally planar lower surface which engages with a second bone surface of a second vertebral body.

7. The containment device of claim 6, wherein the first generally planar upper surface is generally coplanar with a first generally planar upper surface of the cage and the second generally planar lower surface is generally coplanar with a second generally planar lower surface of the cage.

8. The containment device of claim 1, wherein the containment body comprises silicon nitride.

9. The containment device of claim 3, wherein the containment body comprises a hollow structure which contains the second, flowable material therein.

10. The containment device of claim 3, wherein the second, flowable material comprises a silicon nitride putty.
